(19) United States
(12) Patent Application Publication
(10) Pub. No.: US 2017/0128459 A1
(43) Pub. Date: May 11, 2017

(54) PHARMACEUTICAL COMPOSITION CONTAINING SIRT2 INHIBITOR

(71) Applicants: Industrial Cooperation Foundation Chonbuk National University, Jeonju-si, Jeollabuk-do (KR); Chonbuk National University Hospital, Jeonju-si, Jeollabuk-do (KR)

(72) Inventors: Won KIM, Jeollabuk-do (KR); Sung Kwang PARK, Jeollabuk-do (KR); Kyung Pyo KANG, Jeollabuk-do (KR); Yu Jin JUNG, Jeollabuk-do (KR)

(21) Appl. No.: 15/129,664

(22) PCT Filed: May 8, 2014
(86) PCT No.: PCT/KR2014/004108

§ 371 Date: Sep. 27, 2016

(30) Foreign Application Priority Data
Mar. 27, 2014 (KR) 10-2014-0035993
Mar. 27, 2014 (KR) 10-2014-0036032

(51) Int. Cl. A61K 31/55; A23L 33/10; A61K 31/4709

(52) U.S. Cl. CPC A61K 31/55; A61K 31/4709; A23L 33/10; A23V 2002/00

(57) ABSTRACT

The present invention relates to a pharmaceutical composition containing a SIRT2 inhibitor and, more specifically, to: a pharmaceutical composition for preventing or treating renal inflammatory diseases, which are caused by sepsis, by controlling inflammation-inducing factors by sepsis through the regulation of SIRT2 gene expression so as to reduce renal inflammation, thereby preventing a kidney injury; and a pharmaceutical composition for preventing or treating cancer, having an effect of increasing anticancer efficacy while reducing nephrotoxicity, which is a side effect of cisplatin, when administered together with cisplatin.

### TECHNICAL FIELD

The present invention relates to a pharmaceutical composition containing an SIRT2 inhibitor, and more particularly to a pharmaceutical composition for preventing or treating a renal inflammatory disease, which is caused by sepsis, by controlling an inflammation-inducing factor due to sepsis through the regulation of SIRT2 gene expression to reduce renal inflammation, thereby inhibiting kidney injuries; and a pharmaceutical composition for preventing or treating a cancer, the pharmaceutical composition having an effect of enhancing anticancer efficacy while reducing nephrotoxicity, which is a side effect of cisplatin, when administered with cisplatin.

### BACKGROUND ART

Cancers are diseases that become the cause of about 7 million deaths annually worldwide, and, especially in South Korea, since cancers are the leading cause of death by accounting for 23.5% of all causes of death according to the latest [symbol] Cause of Death Statistics Yearbook 2000 [symbol] (the analytical results of the data related to deaths in 2000) by Statistics Korea, cancer control measures at the national level are required. Currently, cancers are treated by various methods, such as surgery, radiation therapy, and gene therapy, but one of the most widely used treatment methods is to administer an anticancer agent.

Anticancer chemotherapy is a systemic treatment, and an anticancer agent that is orally administered or injected mostly spreads throughout the body through the bloodstream. Therefore, anticancer chemotherapy is a treatment acting on micrometastasis throughout a body, rather than having a localized therapeutic effect. Thus, anticancer chemotherapy often has systemic side effects, and, compared to surgery or radiation therapy, the severity of such side effects tends to be high. Although chemotherapy is intended to allow the anticancer agent to take actions selectively against cancer cells by taking advantage of a difference between normal cells and cancer cells in terms of sensitivity to the drug, most anticancer agents have a disadvantage of dose-limiting toxicity by failing to distinguish between normal cells and cancer cells.

Due to the aforementioned problems, targeted anticancer agents have been actively researched. Korean Unexamined Patent Application No. 10-2013-0058631 (publication date: Jun. 4, 2013) is a conventional art related to such targeted anticancer agents and discloses a pharmaceutical composition for inhibiting resistance to a targeted anticancer agent, the pharmaceutical composition containing one or more selected from the group consisting of an integrin β3 neutralizing antibody, integrin β3 siRNA, an Src inhibitor, and Src siRNA as an active ingredient. However, such targeted anticancer agents have a disadvantage of not being able to kill cancer cells. Instead, such targeted anticancer agents are typically drugs that prevent the proliferation and growth of cancer cells by inhibiting elements necessary for cancer cells to grow.

In the meantime, cisplatin (cis-diammine-dichloroplatinum [II]), which is a representative anticancer agent, is widely used for clinical purposes as a chemotherapeutic agent for treating ovarian cancer, bladder cancer, lung cancer, head and neck cancer, testicular cancer, and the like (Rosenberg B., Cancer, 55: pp 2303-2315, 1985). Cisplatin is known to attack cancer cells by generating reactive oxygen species and exhibit anticancer efficacy by inducing inter-intrastrand cross-linking of DNA and the formation of a DNA adduct in cancer cells. However, it is known that side effects such as a loss of hearing, neurotoxicity, and nephrotoxicity occur with a drug concentration equal to or greater than the restricted amount during a therapy (Mollman et al., 1998; Screnci and McKeage, 1999), and that liver toxicity and nephrotoxicity are also observed frequently when cisplatin is administered at a high concentration (Cerosimo R. J., Ann. Pharm., 27: pp 438-441, 1993; Cavalli F. et al., Cancer Treat. Rep., 62: pp 2125-2126, 1978). Such side effects caused by cisplatin are closely related to increased lipid peroxidation due to the reactive oxygen species produced by cisplatin (Matsushima H. et al., J. Lab. Clin. Med., 131: pp 518-526, 1998; Koc A. et al., Mol. Cell Biochem., 278(1-2): pp 79-84, 2005), inhibited antioxidant enzyme activity in tissues (Sadzuka Y. et al., Biochem. Pharmacol., 43: pp 1873-1875, 1992), depleted glutathione (Zhang J. G. and Lindup W. E., Biochem. Pharmcol., 45: pp 2215-2222, 1993), and the collapse of calcium homeostasis in cells (Zhang J. G. and Lindup W. E., Toxicology in Vitro, 10: pp 205-209, 1996).

Recently, effective inhibition of nephrotoxicity due to cisplatin was observed when cisplatin and glutathione ester were administered together (Babu E. et al., Mol. Cell Biochem., 144: pp 7-11, 1995), and inhibiting the toxicity caused by cisplatin through the ingestion of an antioxidant through the diet has gained much attention (Appenroth D. et al., Arch. Toxicol., 71: pp 677-683, 1997).

In contrast, sepsis is defined as conditions in which an infection is confirmed or suspected, and is accompanied by a systemic inflammatory response. Severe sepsis is defined as sepsis accompanied by organ dysfunction (manifested as hypotension, hypoxia, oliguria syndrome, metabolic acidosis, thrombocytopenia, and a consciousness disorder). Septic shock is defined as a case of severe sepsis in which the blood pressure is not normalized even with fluid therapy or the administration of a vasopressor. Sepsis may develop into severe sepsis and ultimately into a clinical stage of septic shock. Clinical sepsis in a broad sense is defined as conditions in which an invasion by a microbial agent is associated with clinical symptoms of an infection. Clinical symptoms of sepsis include, but are not limited to: (1) body temperature >38° C. or <36° C.; (2) heart rate >90 times per minute; (3) breath rate >20 times per minute or PaC02<32 mmHg; (4) white blood cell count >12000/m³, <4,000/cu m³, or >10% immature (band) forms; and (5) an organ dysfunction, excessive agglutination, high blood pressure, or the like.

When an infection occurs, macrophages in the infected area are activated to secrete tumor necrosis factor (TNF)-α and interleukin (IL)-1, thereby causing the amount of plasma proteins released into tissues to increase, the migration of macrophages and lymphocytes to tissues to increase, and the adhesion of platelets to blood vessel walls to increase. In this manner, local blood vessels are blocked, and pathogens are concentrated in the infected area. In particular, sepsis comes with a systemic infection and is accompanied by the serious occlusion of blood vessels as induced by TNF-α. In addition, the systemic release of TNF-α causes a loss of plasma volume due to expanded blood vessels and increased permeability of blood vessels, thereby causing shock. In the case of septic shock, TNF-α brings about blood clots in small blood vessels and the consumption of a large amount of blood coagulation proteins by further triggering the coagulation (blood clotting) within disseminated blood vessels. Since the patient loses his or her blood-clotting ability, important organs such as kidney, liver, heart, and lungs are damaged due to a dysfunction of normal perfusion. The mortality rate of severe sepsis and septic shock has been reported to be as high as 25 to 30% and 40 to 70%, respectively.

Although E. coli is the pathogen in various cases of sepsis, other Gram-negative bacteria such as the Klebsiella-Enterobacter-Serratia group and Pseudomonas may also initiate such conditions. Gram-positive microorganisms, such as Staphylococcus, and systemic viral and fungal infections also initiate sepsis in some cases.

It has been understood that sepsis is caused as a result of complex interactions among a causative organism of the infection, the immunity of the host, inflammations, and a coagulation system. Both the response intensity of the host and characteristics of the causative organism greatly affect the prognosis of sepsis. Organ dysfunction observed with sepsis occurs when the host responds inappropriately to the causative organism of the infection, and, if the host's response to the causative organism is too intense, the response may cause damage to an organ of the host itself. Based on this concept, antagonists against TNF-α, IL-1β, IL-6, and the like, which are proinflammatory cytokines playing a leading role in the inflammatory responses of the host, have been tried as a treatment for sepsis but have only failed in most cases. Also, mechanical ventilation therapy, the administration of activated protein C, glucocorticoid therapy, and the like are currently being attempted, but several limitations thereof are being indicated. In addition, a lack of research and treatment methods of sepsis, effective treatments of which are yet to be developed despite a high mortality rate, and inflammatory diseases caused by sepsis has been a problem.

In the meantime, sirtuin-2 (SIRT2) is a member of the sirtuin protein family, and plays a role in important cell survival functions under certain conditions. However, biological functions of the SIRT2 protein related to inflammation and oxidative stress are not clearly known.

SIRT2 (silent information regulator-2), which is known as a sirtuin, is an NAD+-dependent deacetylase regulator in biological processes such as life, aging, cancer initiation, neurodegeneration, and metabolic diseases (Michan, S.; Sinclair, D. Biochem. J. 404: 1-13; 2007, Finkel, T. et al. Nature 460: 587-591; 2009, Donmez, Z.; Guarente, L. Aging Cell 9: 285-290; 20101-3). The SIRT2 gene family is highly conserved from bacteria to eukaryotes. In humans, seven types of SIRTs have been discovered (Frye, R. A. Biochem. Biophys. Res. Comm. 272: 793-798; 2000). Among the seven types of SIRTs in humans, most studies are focused on SIRT1. SIRT1 overexpression increases cell viability under DNA damage and oxidative stress. Also, the neuroprotective role of SIRT1 is well established in Alzheimer's disease and amyotrophic lateral sclerosis. However, biological functions and mechanisms of SIRT2 related to inflammation and oxidative stress are not clearly known.

In the meantime, many chemotherapeutic agents including cisplatin are known to attack cancer cells by generating reactive oxygen species, and the generated reactive oxygen species are known to act on normal cells, thereby damaging the same. Therefore, substances having an antioxidative effect are likely to reduce the toxicity caused by a chemotherapeutic agent. Also, it is believed that liver toxicity and nephrotoxicity caused by an external toxic substance generating reactive oxygen species or radicals can also be effectively inhibited, but the ways to realize such inhibition have not been thoroughly researched.

In addition, as mentioned above, biological functions and mechanisms of SIRT2 related to inflammation and oxidative stress have not been thoroughly researched, and molecular mechanisms that are related to renal inflammatory diseases caused by sepsis, and to the intracellular interactions, signal transduction, and regulatory mechanisms of SIRT2 have been scarcely studied such that the methods capable of preventing and treating renal inflammatory diseases caused by sepsis are scarce.

### DISCLOSURE

### Technical Problem

The present invention was devised to solve the aforementioned problems. First, the present invention is directed to providing a pharmaceutical composition for preventing and treating a nephrotoxic disease caused by an anticancer agent, wherein, upon administering of the anticancer agent, the pharmaceutical composition inhibits nephrotoxicity caused by the anticancer agent while enhancing anticancer efficacy.

Second, the present invention is directed to providing a pharmaceutical composition for preventing and treating an inflammatory disease.

Third, the present invention is directed to providing a pharmaceutical composition for preventing and treating a nephrotoxic disease caused by an anticancer agent, wherein, upon administering of the anticancer agent, the pharmaceutical composition inhibits nephrotoxicity by the anticancer agent while enhancing anticancer efficacy.

Fourth, the present invention is directed to providing an anticancer adjuvant having an inhibitory activity on the nephrotoxicity caused by an anticancer agent.

Fifth, the present invention is directed to providing a health functional food for preventing and improving a nephrotoxic disease caused by an anticancer agent.

### Technical Solution

The present invention provides a pharmaceutical composition for preventing and treating a nephrotoxic disease caused by an anticancer agent, the pharmaceutical composition containing an SIRT2 inhibitor as an active ingredient.

According to an exemplary embodiment of the present invention, the anticancer agent may be cisplatin.

According to another exemplary embodiment of the present invention, the SIRT2 inhibitor may be an antisense oligonucleotide, siRNA, an aptamer, or an antibody specific to the SIRT2 gene.

According to still another exemplary embodiment of the present invention, the SIRT2 inhibitor may be AGK2 or AK-1.

According to yet another exemplary embodiment of the present invention, the pharmaceutical composition may inhibit kidney injuries by inhibiting the expression of ICAM-1 and VCAM-1, which are molecules related to apoptosis and inflammatory response.

In addition, the present invention provides an anticancer adjuvant containing, as an active ingredient, an SIRT2 inhibitor that has an inhibitory activity with respect to nephrotoxicity caused by an anticancer agent.

Further, the present invention provides a health functional food for preventing and treating a nephrotoxic disease caused by an anticancer agent, the health functional food containing an SIRT2 inhibitor as an active ingredient.

Furthermore, the present invention provides a pharmaceutical composition for preventing and treating an inflammatory disease, the pharmaceutical composition containing an SIRT2 inhibitor as an active ingredient.

According to an exemplary embodiment of the present invention, the inflammatory disease may be a renal inflammatory disease caused by sepsis.

According to another exemplary embodiment of the present invention, the SIRT2 inhibitor may be one or more selected from the group consisting of an antisense oligonucleotide, siRNA, an aptamer, or an antibody specific to the SIRT2 gene.

According to still another exemplary embodiment of the present invention, the SIRT2 inhibitor may be AGK2 or AK-1.

According to yet another exemplary embodiment of the present invention, the pharmaceutical composition may inhibit kidney injuries by reducing renal inflammation by inhibiting the expression of CXCL2 and CCL2, which are LPS-induced inflammation-inducing factors.

Moreover, the present invention provides a health functional food for preventing and improving an inflammatory disease, the health functional food containing an SIRT2 inhibitor as an active ingredient.

Further, the present invention provides a pharmaceutical composition for kidney protection, the pharmaceutical composition containing an SIRT2 inhibitor as an active ingredient.

In addition, the present invention provides a health functional food for kidney protection, the health functional food containing an SIRT2 inhibitor as an active ingredient.

### Advantageous Effects

By inhibiting kidney injuries by reducing renal inflammation by inhibiting the expression of CXCL2 and CCL2, which are LPS-induced inflammation-inducing factors, the SIRT2 inhibitor of the present invention can be usefully employed as a pharmaceutical composition and a health functional food for preventing and treating a renal inflammatory disease caused by sepsis.

In addition, the SIRT2 inhibitor according to the present invention is found to be highly effective for inhibiting kidney injuries caused by cisplatin, which is an anticancer agent, reducing nephrotoxicity, and enhancing anticancer efficacy by inhibiting apoptosis and regulating the expression of ICAM-1 and VCAM-1, which are factors related to inflammatory response. Also, when used together with an anticancer agent, the SIRT2 inhibitor according to the present invention is found to enhance the anticancer efficacy of the anticancer agent. Therefore, the SIRT2 inhibitor according to the present invention can be usefully employed as a pharmaceutical composition or a health functional food for preventing and treating a nephrotoxic disease caused by an anticancer agent.

### DESCRIPTION OF DRAWINGS

FIGS. 1 to 4 provide the results of examining an effect on CXCL2 expression, which is regulated by an LPS, in an SIRT2-gene knockout mouse (SIRT2+/+: an experimental group in which SIRT2 is present; SIRT2−/−: an experimental group lacking SIRT2; CB: a control group administered a control buffer; LPS: a control group administered the LPS).

FIG. 1 is a set of images for observing CXCL2 expression in a mouse kidney through immunochemical staining,

FIG. 2 is a graph providing the result of determining, by an image analysis program, the density of CXCL2-positive cells observed through staining,

FIG. 3 is a graph providing the result of determining the CXCL2 level in a mouse serum through an enzyme linked immunosorbent assay, and

FIG. 4 is a graph providing the result of determining the CXCL2 level in mouse kidney tissues through an enzyme linked immunosorbent assay.

FIGS. 5 to 8 provide the results of examining an effect on CCL2 expression, which is regulated by an LPS, in an SIRT2-gene knockout mouse (SIRT2+/+: an experimental group in which SIRT2 is present; SIRT2−/−: an experimental group lacking SIRT2; CB: a control group administered a control buffer; LPS: a control group administered the LPS).

FIG. 5 is a set of images for observing CCL2 expression in a mouse kidney through immunochemical staining,

FIG. 6 is a graph providing the result of determining, by an image analysis program, the density of CCL2-positive cells observed through staining,

FIG. 7 is a graph providing the result of determining the CCL2 level in a mouse serum through an enzyme linked immunosorbent assay, and

FIG. 8 is a graph providing the result of determining the CCL2 level in mouse kidney tissues through an enzyme linked immunosorbent assay.

FIGS. 9 to 14 provide the results of examining CXCL2 expression patterns, which are affected by regulated SIRT2 gene expression, in kidney cells (SIRT2 siRNA: an experimental group treated with SIRT2 siRNA; cont siRNA: a control group treated with siRNA; ad SIRT2: an experimental group treated with an SIRT2 recombinant adenovirus; ad cont: a control group treated with a control virus; CB: a control group administered a control buffer; LPS: a control group administered an LPS).

FIG. 9 is a graph providing the result of examining an mRNA expression pattern of CXCL2 through qRT-PCR when the SIRT2 in mouse proximal tubule cells was knocked out using siRNA,

FIG. 10 is a graph providing the result of determining the CXCL2 level in a cell culture medium through an enzyme linked immunosorbent assay,

FIG. 11 is a graph providing the result of determining the CXCL2 level in cells through an enzyme linked immunosorbent assay, and

FIG. 12 is a graph providing the result of examining an mRNA expression pattern of CXCL2 through qRT-PCR when the SIRT2 in mouse proximal tubule cells was increased using an adenovirus.

FIG. 13 is a graph providing the result of determining the CXCL2 level in a cell culture medium through an enzyme linked immunosorbent assay, and

FIG. 14 is a graph providing the result of determining the CXCL2 level in cells through an enzyme linked immunosorbent assay.

FIGS. 15 to 20 provide the results of examining the CCL2 expression patterns, which are affected by regulated SIRT2 gene expression, in kidney cells (SIRT2 siRNA: an experimental group treated with SIRT2 siRNA; cont siRNA: a control group treated with siRNA; ad SIRT2: an experimental group treated with an SIRT2 recombinant adenovirus; ad cont: a control group treated with a control virus; CB: a control group administered a control buffer; LPS: a control group administered an LPS).

FIG. 15 is a graph providing the result of examining an mRNA expression pattern of CCL2 through qRT-PCR when the SIRT2 in mouse proximal tubule cells was knocked out using siRNA,

FIG. 16 is a graph providing the result of determining the CCL2 level in a cell culture medium through an enzyme linked immunosorbent assay,

FIG. 17 is a graph providing the result of determining the CCL2 level in cells through an enzyme linked immunosorbent assay, and

FIG. 18 is a graph providing the result of examining an mRNA expression pattern of CCL2 through qRT-PCR when the SIRT2 in mouse proximal tubule cells was increased using an adenovirus.

FIG. 19 is a graph providing the result of determining the CCL2 level in a cell culture medium through an enzyme linked immunosorbent assay, and

FIG. 20 is a graph providing the result of determining the CCL2 level in cells through an enzyme linked immunosorbent assay.

FIGS. 21 and 22 are graphs providing the result of examining, through qRT-PCR, the expression patterns of CXCL2 (FIG. 22) and CCL2 (FIG. 23), which are affected by an SIRT2 inhibitor, in kidney cells.

FIGS. 23 and 24 are graphs providing the result of determining a degree of kidney injuries, which is affected by SIRT2 gene expression, upon LPS administration, by measuring the levels of neutrophil gelatinase-associated lipocalin (NGAL) (FIG. 23) and a kidney injuries molecule (KIM-1) (FIG. 24) through an enzyme linked immunosorbent assay.

FIG. 25 is a graph providing the result of measuring BUN for determining the kidney functions affected by cisplatin in an SIRT2-gene knockout mouse.

FIG. 26 is a graph providing the result of measuring creatinine for determining the kidney functions affected by cisplatin in an SIRT2-gene knockout mouse.

FIG. 27 a set of images for observing, through PAS staining, histological damage to a kidney caused by the administration of cisplatin.

FIG. 28 is a graph providing the result of determining the survival rate affected by the administration of cisplatin.

FIG. 29 provides the result of the western blot for determining, based on a caspase-3 expression pattern, an effect of SIRT2 gene expression on controlling apoptosis. In FIG. 29, “WT” refers to a control group in which SIRT2 is present, and “KO” refers to an SIRT2 knockout control group. The first lane corresponds to a mouse having SIRT2 and treated with a vehicle, the second lane is an SIRT2 knockout mouse treated with a vehicle, the third lane to sixth lane correspond to mice having SIRT2 and treated with cisplatin, and the seventh lane to tenth lane correspond to SIRT2 knockout mice treated with cisplatin. The expression of an active form of caspase-3, which was not observed in the control groups treated only with a vehicle, was found to increase in the mouse having SIRT2 upon treatment with cisplatin and significantly decrease in the SIRT2 knockout mouse.

FIG. 30 provides the result of the western blot for determining, based on a p53 expression pattern, an effect of SIRT2 gene expression on controlling apoptosis. p53 acetylation, which increased in the mouse having SIRT2 upon the treatment with cisplatin, was found to decrease in the SIRT2 knockout mouse.

FIG. 31 provides the result of the western blot for determining, based on ICAM-1 and VCAM-1 expression patterns, an effect of SIRT2 gene expression on controlling inflammatory molecules. In the first and second lanes, ICAM-1 and VCAM-1 were not expressed upon treatment with a vehicle regardless of the presence of SIRT2. In the third lane to fifth lane, an increase in the expression of ICAM-1 and VCAM-1 was observed in the mice that have SIRT2 and were treated with cisplatin. However, in sixth lane to eighth lane, which correspond to SIRT2 knockout control group mice, the expression of ICAM-1 and VCAM-1 upon treatment with cisplatin was lower compared to the control group mice having SIRT2.

FIG. 32 is a set of cell images for observing an effect of SIRT2 inhibitor on inhibiting cell damage caused by cisplatin.

FIG. 33 is a graph providing the quantified result of FIG. 32.

FIG. 34 is a graph providing the result of examining a cell proliferation effect of an SIRT2 inhibitor through a cell proliferation test by performing XTT.

### BEST MODES OF THE INVENTION

Hereinafter, the present invention will be described in greater detail.

Since the blood-clotting ability of a patient is lost due to sepsis as described above, important organs such as the kidney, liver, heart, and lungs are damaged due to a dysfunction of normal perfusion. Despite the high mortality rate when organs such as a kidney is affected by sepsis, effective treatments of sepsis are yet to be developed, and the lack of research and treatment methods of sepsis and inflammatory diseases caused by sepsis has been a problem.

In addition, biological functions and mechanisms of SIRT2 related to inflammation and oxidative stress have not been thoroughly studied, and, since molecular mechanisms that are related to nephrotoxic diseases caused by cisplatin, which is a representative anticancer agent, and to the intracellular interaction, signal transduction, and regulatory mechanisms of SIRT2 have been scarcely studied such that the methods capable of preventing and treating a nephrotoxic disease caused by an anticancer agent are scarce.

Hence, the present invention aims to solve the aforementioned problems by providing a pharmaceutical composition for preventing and treating an inflammatory disease, the pharmaceutical composition containing an SIRT2 inhibitor as an active ingredient; and a pharmaceutical composition for preventing and treating a nephrotoxic disease caused by an anticancer agent, the pharmaceutical composition containing an SIRT2 inhibitor as an active ingredient. By controlling inflammation-inducing factors caused by sepsis through the regulation of SIRT2 gene expression to reduce renal inflammation and thereby inhibiting kidney injuries, the pharmaceutical composition of the present invention can be usefully employed as a pharmaceutical composition for preventing and treating a renal inflammatory disease caused by sepsis.

The present invention contains an SIRT2 inhibitor as an active ingredient.

In the present invention, “SIRT2 (Sirtuin 2)” is a member of the sirtuin protein family, and plays a role in important cell survival functions under certain conditions.

SIRT2 (silent information regulator-2), which is known as a sirtuin, is an NAD+-dependent deacetylase regulator in biological processes such as life, aging, cancer initiation, neurodegeneration, and metabolic diseases (Michan, S.; Sinclair, D. Biochem. J. 404: 1-13; 2007, Finkel, T. et al. Nature 460: 587-591; 2009, Donmez, Z.; Guarente, L. Aging Cell 9: 285-290; 20101-3). The SIRT2 gene family is highly conserved from bacteria to eukaryotes. In humans, seven types of SIRTs have been discovered (Frye, R. A. Biochem. Biophys. Res. Comm. 272: 793-798; 2000). Among the seven types of SIRTs in humans, most studies are focused on SIRT1. SIRT1 overexpression increases cell survival under DNA damage and oxidative stress (Oberdoerffer, P. et al. Cell 135: 907-918; 2008). Also, the neuroprotective role of SIRT1 is well established in Alzheimer's disease and amyotrophic lateral sclerosis (Chen, J. et al. J. Biol. Chem. 280: 40364-40374; 2005, Kim, D. et al. EMBO J. 26: 3169-3179; 2007). However, biological functions and mechanisms of SIRT2 related to inflammation and oxidative stress are not clearly known.

Therefore, by revealing the molecular mechanisms that are related to renal inflammatory diseases caused by sepsis, and to the intracellular interactions, signal transduction, and regulatory mechanisms of SIRT2, the present invention provides a pharmaceutical composition for preventing and treating a renal inflammatory disease caused by sepsis.

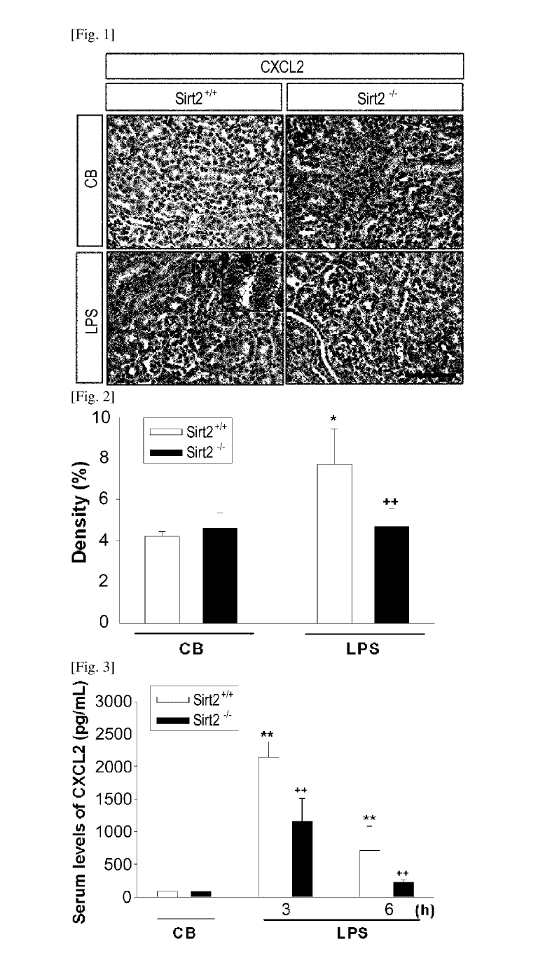

Specifically, as can be learned through Example 3 and Example 4, the expression of CXCL2 and CCL2 was determined by performing immunochemical staining and an enzyme linked immunosorbent assay on mouse kidney tissues to examine an effect on regulating the expression of CXCL2 and CCL2, which had been increased by an LPS, in an SIRT2-gene knockout mouse, and the expression of CXCL2 and CCL2 in blood was also determined through an enzyme linked immunosorbent assay. Based on the result, it can be learned that the expression of CXCL2 and CCL2, which had increased upon the administration of the LPS, decreased in the SIRT2-gene knockout mouse (see FIGS. 1 to 8).

It can be learned that the expression of CXCL2 and CCL2, which had been increased by the administration of an LPS, decreased in the SIRT2-gene knockout mouse (see FIGS. 1 to 8).

In addition, the anticancer agent of the present invention may be cisplatin.

Cisplatin (cis-diammine-dichloroplatinum [II]), which is a representative anticancer agent, is widely used for clinical purposes as a chemotherapeutic agent for treating ovarian cancer, bladder cancer, lung cancer, head and neck cancer, testicular cancer, and the like (Rosenberg B., Cancer, 55: pp 2303-2315, 1985). Cisplatin is known to attack cancer cells by generating reactive oxygen species and exhibit anticancer efficacy by inducing inter-intrastrand cross-linking of DNA and the formation of DNA adducts in cancer cells. However, it is known that side effects such as a loss of hearing, neurotoxicity, and nephrotoxicity occur with a drug concentration equal to or greater than the restricted amount during a therapy (Mollman et al., 1998; Screnci and McKeage, 1999), and that liver toxicity and nephrotoxicity are also observed frequently when cisplatin is administered at a high concentration.

Therefore, by revealing the molecular mechanisms that are related to nephrotoxic diseases caused by an anticancer agent, and to the intracellular interactions, signal transduction, and regulatory mechanisms of SIRT2, the present invention provides a pharmaceutical composition for preventing and treating a nephrotoxic disease caused by an anticancer agent.

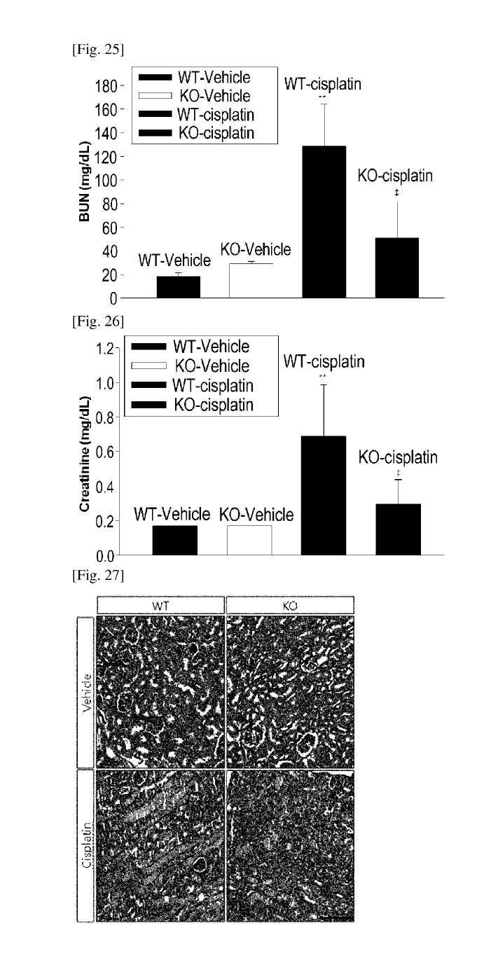

Specifically, as can be learned through Example 10, cisplatin was administered to an SIRT2-gene knockout mouse and the kidney functions and kidney damage of the mouse were assessed to examine an effect of SIRT2 gene expression on nephrotoxicity caused by cisplatin. Based on the results, it can be learned that the administration of cisplatin to the mouse caused kidney injuries, and that BUN and creatinine, which are measurement standards of kidney injuries, increased accordingly. Also, it was found that BUN and creatinine, which had increased by cisplatin, significantly decreased in an SIRT2-gene knockout mouse (see FIGS. 25 and 26).

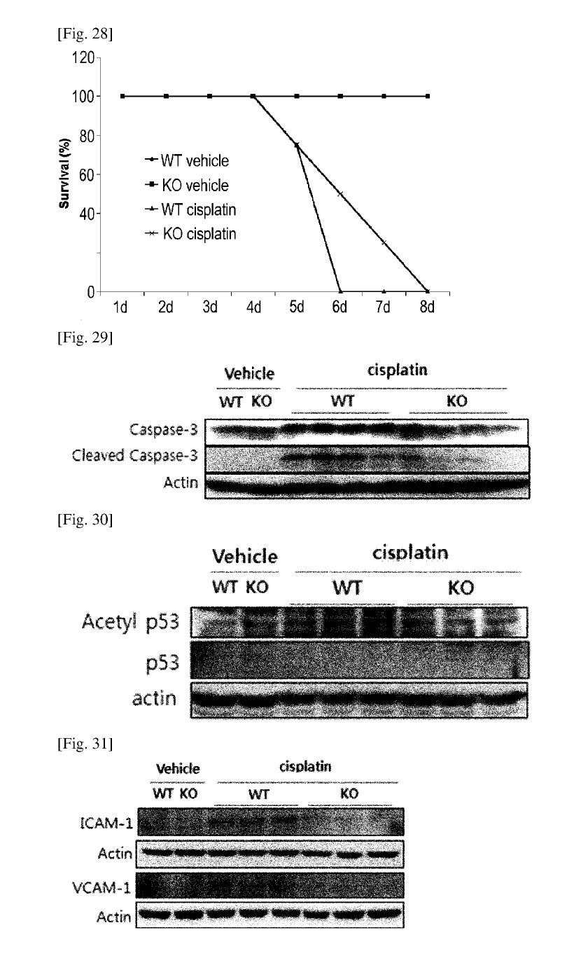

In addition, PAS staining was performed to examine histological damage to a kidney as a result of administration of cisplatin, and, as shown in FIG. 27, the results showed that tissue damage such as the detachment of epithelial cells, a loss of a brush border, and the formation of a tubular cast were observed in the kidney of the control group of SIRT2 gene WT (SIRT2+/+) mice administered cisplatin, whereas such kidney injuries were significantly lower in the SIRT2-gene knockout (SIRT2−/−) mouse. Not only that, the survival rate also increased (see FIG. 28).

Based on these results, it can be learned that kidney injuries caused by cisplatin are inhibited when an SIRT2 gene is knocked out.

Unless defined otherwise, the term “the treatment” used in the present invention refers to reversing, reducing, inhibiting the progression of, or preventing an aforementioned disease or disorder, or one or more symptoms of the aforementioned disease or disorder, and the term “the treatment” used in the present invention refers to an act of treating when treatment is defined as described above.

Therefore, by inhibiting kidney injuries by reducing renal inflammation by inhibiting the expression of CXCL2 and CCL2, which are LPS-induced inflammation-inducing factors, in an SIRT2-gene knockout mouse, the SIRT2 inhibitor of the present invention can be usefully employed as a pharmaceutical composition for preventing and treating a renal inflammatory disease caused by sepsis.

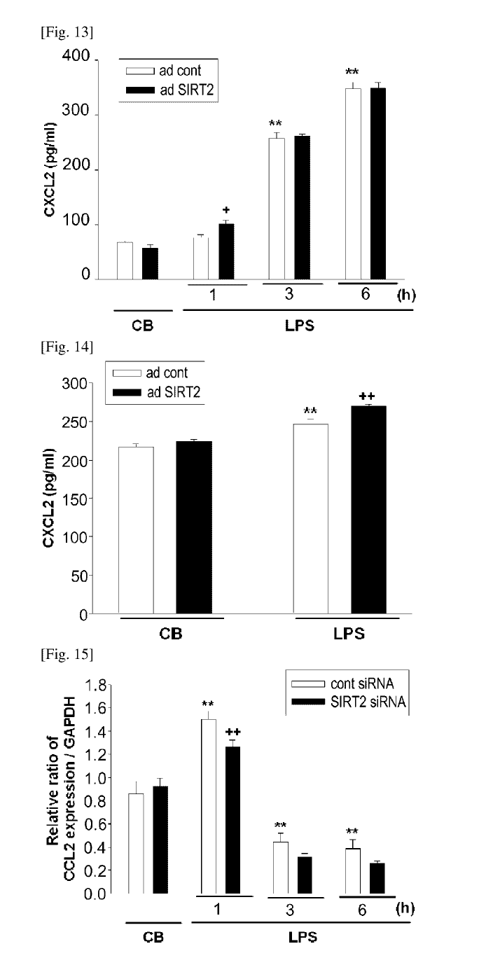

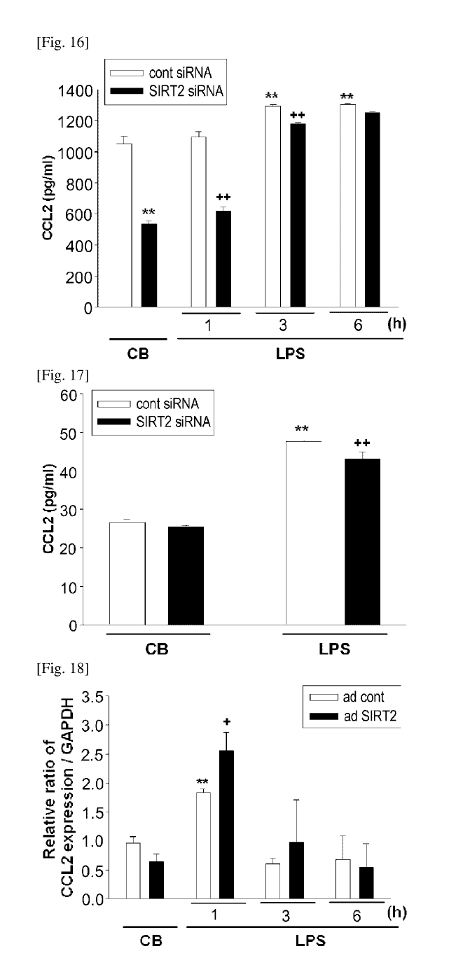

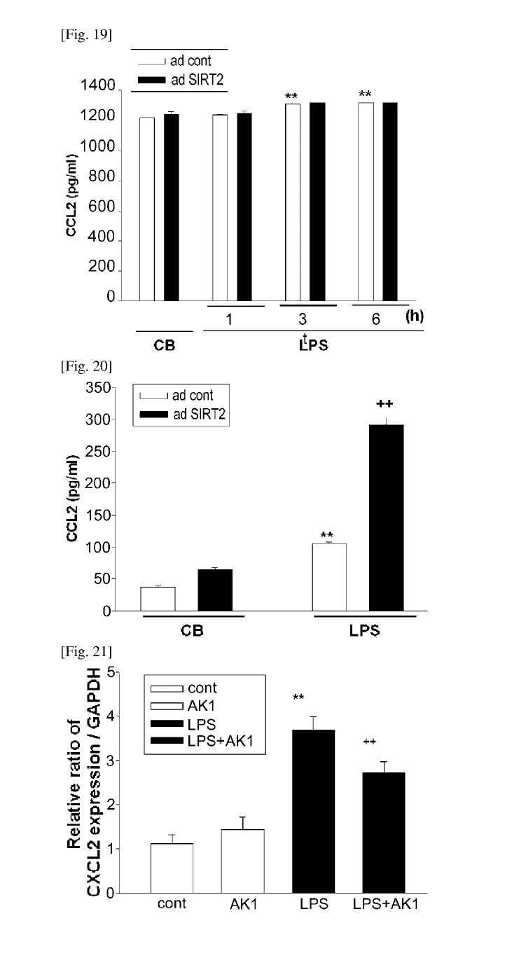

Specifically, as shown in Example 5 and Example 6, when an effect on CXCL2 and CCL2 expression (which is increased by an LPS) by knocking out an SIRT2 gene in mouse proximal tubule cells using siRNA was examined when the SIRT2-gene knockout cells were treated with an LPS, the results showed a decrease in the mRNA expression of CXCL2 and CCL2, which had been increased by an LPS. Conversely, when an SIRT2-recombinant adenovirus was prepared and was used for SIRT2 gene overexpression, a significant increase in the expression of CXCL2 and CCL2 by an LPS was observed (see FIGS. 9 to 20).

Based on these results, it can be learned that SIRT2 gene expression affects the expression of CXCL2 and CCL2, which are inflammation-inducing factors increased by treatment with an LPS.

Further, the SIRT2 inhibitor according to the present invention is found to be highly effective for inhibiting kidney injuries caused by cisplatin, which is an anticancer agent, reducing nephrotoxicity, and enhancing anticancer efficacy by inhibiting apoptosis through the regulation of the p53 acetylation pathway and reducing the expression of ICAM-1 and VCAM-1, which are factors related to inflammatory response. Also, when used together with an anticancer agent, the SIRT2 inhibitor according to the present invention is found to enhance the anticancer efficacy of the anticancer agent. Therefore, the SIRT2 inhibitor according to the present invention can be usefully employed as a pharmaceutical composition for preventing and treating a nephrotoxic disease caused by an anticancer agent.

Specifically, as can be learned through Example 10, since kidney injuries caused by cisplatin are generally affected by apoptosis, the acetylation of caspase-3 and p53 was examined through western blotting to assess an effect of SIRT2 gene knockout on controlling apoptosis caused by cisplatin. The results showed that cleaved caspase-3, which increased in the group of WT mice administered cisplatin, significantly decreased in an SIRT2-gene knockout mouse, in which the expression of “acetyl p53” also decreased (see FIGS. 29 and 30).

Such results indicate reduced apoptosis in kidney tissues of an SIRT2-gene knockout mouse, which was found to be controlled through the p53 acetylation pathway.

Further, as can be learned through Example 12, when an investigation was performed to see if the expression of ICAM-1 and VCAM-1 genes, which are molecules related to inflammatory responses caused by cisplatin, is regulated by an SIRT2 gene, it was found that the expression of ICAM-1 and VCAM-1 genes, which increased upon the administration of cisplatin to a WT mouse, decreased when cisplatin was administered to an SIRT2-gene knockout mouse (see FIG. 31).

The pharmaceutical composition according to the present invention may contain a pharmaceutically effective amount of an SIRT inhibitor either alone or together with one or more pharmaceutically acceptable carriers, excipients, or diluents.

In the above description, a “pharmaceutically effective amount” refers to an amount sufficient for preventing, improving, and treating the symptoms of an inflammatory disease or an amount sufficient for preventing, improving, and treating the symptoms of a nephrotoxic disease caused by an anticancer agent.

The pharmaceutically effective amount of the SIRT2 inhibitor according to the present invention is 0.5 to 100 mg/kg/day, and is preferably 0.5 to 5 mg/kg/day. However, the pharmaceutically effective amount may be suitably changed depending on the severity of symptoms of a nephrotoxic disease, the age, weight, health, and sex of the patient, the route of administration, the duration of treatment, and the like.

Also, in the above description, “pharmaceutically acceptable” has a meaning that the composition is physiologically acceptable and typically not causing an allergic reaction, such as a gastrointestinal disorder and dizziness, or a similar reaction when administered to a human. Examples of the carriers, excipients, and diluents may include lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oils. Also, a filler, anti-coagulant, lubricant, wetting agent, flavoring agent, emulsifier, preservative, and the like may be additionally included in the composition.

In addition, the composition of the present invention may be formulated using a method well known in the art to provide a quick, sustained, or delayed release of the active ingredient after being administered to a mammal. The formulation may be in a form of a powder, granule, tablet, emulsion, syrup, aerosol, soft or hard gelatin capsule, sterile injectable solution, or sterile powder.

Moreover, the pharmaceutical composition according to the present invention may be administered through various routes including oral, dermal, subcutaneous, intravenous, and intramuscular administration, and the dose of the active ingredient(s) may be suitably selected depending on various factors such as the route of administration, the age, sex, and weight of the patient, the severity of the patient's condition, and the like. The composition for preventing or treating an inflammatory disease according to the present invention may be administered together with a well-known compound effective for preventing, improving, or treating the symptoms of the inflammatory disease.

Further, in the present invention, the SIRT2 inhibitor includes, but is not limited to: an antisense oligonucleotide, RNAi, siRNA, shRNA, aptamer, antibody, single chain variable region fragment (scFv), low-molecular-weight compound, or natural extract that is specific to the SIRT2 gene, and is preferably AGK2 or AK-1, and more preferably AK-1.

The AK-1 may include a compound represented by the following Structural Formula 1.

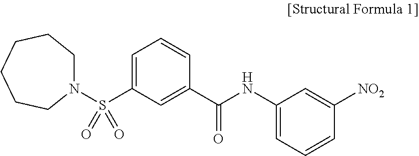 [SMILES: O=C(Nc1cccc([N+](=O)[O-])c1)c1cccc(S(=O)(=O)N2CCCCCC2)c1]

The AGK2 may include a compound represented by the following Structural Formula 2.

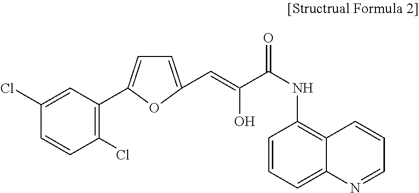 [SMILES: O=C(Nc1cccc2ncccc12)/C(O)=C/c1ccc(-c2cc(Cl)ccc2Cl)o1]

In addition, the AK-1 and/or the AGK2 may be able to inhibit SIRT2 activity by targeting an SIRT2 nicotinamide binding site through the cell penetration of benzylsulfonamide.

The SIRT2 inhibitor of the present invention provides an inhibitory effect on LPS-induced renal inflammatory responses and kidney functional damage that are caused by sepsis.

Specifically, as can be learned through Example 7, mouse proximal tubule cells were treated with AK-1 (10 μM), which is an SIRT2 inhibitor, 30 minutes prior to being treated with an LPS (10 μg/mL) for 1 hour, and were subsequently collected for determining the mRNA expression levels of CXCL2 and CCL2 through qRT-PCR to determine the expression patterns of CXCL2 and CCL2, which are inflammation-inducing factors, that are affected by an SIRT2 inhibitor in kidney cells. Based on the results, it was found that the expression of CXCL2 and CCL2, which had been increased by the LPS, decreased following the treatment of AK-1, which is an SIRT2 inhibitor (see FIGS. 21 and 22).

In addition, as can be learned through Example 8, a mouse was treated with an LPS, and the urine of the mouse was collected from the bladder 3 hours later to determine a degree of kidney functional damage. The urine that had been collected was subjected to an enzyme linked immunosorbent assay to determine the levels of NGAL and KIM-1.

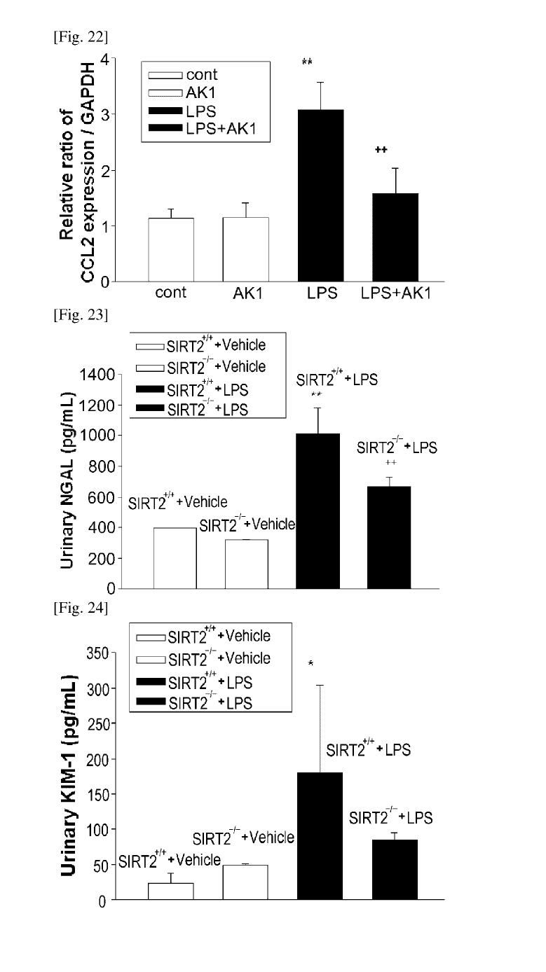

The result shows that the levels of NGAL and KIM-1, which increased in the control group administered the LPS, significantly decreased in an SIRT2-gene knockout mouse (FIGS. 23 to 24).

Based on the result, it can be found that the SIRT2 gene also affects the kidney injuries that have been induced by an LPS, and that the kidney injuries caused by an LPS can be inhibited by the regulation of the SIRT2 gene.

Further, the SIRT2 inhibitor of the present invention provides an effect of inhibiting cell damage caused by cisplatin, which is an anticancer agent, and of cell proliferation.

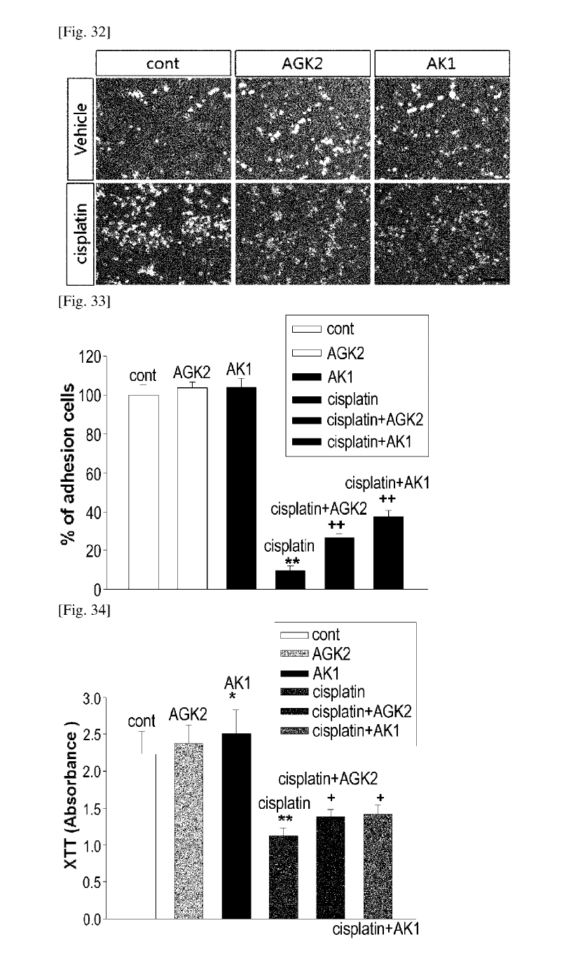

Specifically, as can be learned through the Example 13 of the present invention, mouse proximal tubule cells were treated with cisplatin and the effects of AGK2 and AK-1, which are SIRT2 inhibitors, were observed to examine an effect of an SIRT2 inhibitor on inhibiting cell damage caused by cisplatin. Based on the result, it can be found that the number of adhesion cells decreased in the cells administered cisplatin, whereas the number of adhesion cells significantly increased in the control group treated with an SIRT2 inhibitor (see FIGS. 32 to 33).

Moreover, when the cell proliferation efficacy of an SIRT2 inhibitor was assessed through a cell proliferation test, as shown in FIG. 34, the result of an XTT test shows that the cell proliferation, which decreased upon treatment with cisplatin, significantly increased when either AGK2 or AK-1, both which are SIRT2 inhibitors, was also used provided.

Based on these results, it can be found that knocking out an SIRT2 gene is effective for kidney protection by inhibiting kidney injuries caused by cisplatin, which suggests that the regulation of SIRT2 gene expression is effective for treating acute kidney injuries.

In the present invention, the term an “antisense oligonucleotide” refers to DNA or RNA including a nucleic acid sequence complementary to a particular mRNA sequence, or a derivative of the DNA or RNA, and such an antisense oligonucleotide functions to bind to the complementary mRNA sequence to inhibit the translation of the mRNA into a protein. In the present invention, an “antisense sequence” refers to a DNA or RNA sequence that is complementary to the mRNA of the aforementioned gene and is capable of binding to the mRNA, and such an antisense sequence can inhibit activity that is essential for the translation, translocation into a cytoplasm, or maturation of the mRNA, or for all other overall biological functions.

Moreover, the antisense nucleic acid may be modified at one or more base, sugar, or backbone positions to enhance efficacy (De Mesmaeker et al., Curr Opin Struct Biol., 5, 3, 343-55, 1995). The nucleic acid backbone may be modified into a phosphorothioate linkage, a phosphotriester linkage, a methylphosphonate linkage, a short-chain alkyl intersugar linkage, a cycloalkyl intersugar linkage, a short-chain heteroatomic intersugar linkage, a heterocyclic intersugar linkage, or the like. Also, the antisense nucleic acid may include one or more substituted sugar moieties. The antisense nucleic acid may include a modified base. Examples of the modified base include hypoxanthine, 6-methyladenine, 5-methylpyrimidine (5-methylcytosine, in particular), 5-hydroxymethylcytosine (HMC), glycosyl HMC, gentobiosyl HMC, 2-aminoadenine, 2-thiouracil, 2-thiothymine, 5-bromouracil, 5-hydroxymethyluracil, 8-azaguanine, 7-deazaguanine, N6(6-aminohexyl)adenine, and 2,6-diaminopurine. In addition, the antisense nucleic acid of the present invention may be chemically bonded to one or more moieties or conjugates that enhance the antisense nucleic acid activity and cell adhesion characteristics. Examples of the moieties include, but are not limited to: lipophilic moieties such as cholesterol moieties, cholesteryl moieties, cholic acid, thioethers, thiocholesterol, aliphatic chains, phospholipids, polyamine, polyethylene glycol chains, adamantane acetic acid, palmityl moieties, octadecylamine, and hexylamino-carbonyl-oxycholesterol moieties. Oligonucleotides including a lipophilic moiety and the methods of preparing such oligonucleotides are already well known in the technical field of the present invention (U.S. Pat. Nos. 5,138,045; 5,218,105; and 5,459,255). The modified nucleic acid may enhance stability against nuclease activity and increase binding affinity between the antisense nucleic acid and the targeted mRNA.

The antisense oligonucleotide may be synthesized in vitro by a conventional method and then administered into a living body, or an in vivo synthesis of the antisense oligonucleotide may be induced. One example of synthesizing the antisense oligonucleotide in vitro involves the use of RNA polymerase I. One example of synthesizing the antisense RNA in vivo involves the use of a vector that has an origin of a recognition site (MCS) in an opposite direction to transcribe antisense RNA. Such antisense RNA preferably includes a translation stop codon within the sequence thereof so that it is not translated into a peptide sequence.

In the present invention, “RNAi” refers to RNA interference, and has a meaning of RNA interference. RNA interference is a specific gene inhibition phenomenon well preserved among most organisms. RNA interference is considered as a kind of a gene monitoring mechanism that a cell uses as a defensive measure against viral infection, or to inhibit transposon activity or remove abnormal mRNA. In particular, the gene inhibition phenomenon caused by small RNA is referred to as RNA interference in a broad sense, and the RNA interference refers to a phenomenon of mRNA degradation caused by siRNA in a narrow sense. In addition, RNA interference also refers to a gene suppression experimental technique using siRNA.

In the present invention, the term “siRNA” refers to a nucleic acid molecule capable of mediating RNA interference or gene silencing (refer to International Patent Publication No. 00/44895, 01/36646, 99/32619, 01/29058, 99/07409, and 00/44914). Being capable of inhibiting the expression of the target gene, siRNA is provided as an effective gene knockdown method or a gene treatment method.

The siRNA molecule of the present invention may have a double-chain structure in which one of a sense strand (a sequence that corresponds to the mRNA sequence of the marker gene) and an antisense strand (a sequence that is complementary to the mRNA sequence) is positioned on the opposite side of the other. Also, the siRNA molecule of the present invention may have a single-chain structure having a self-complementary sense strand and a self-complementary antisense strand. Further, siRNA is not limited to the double-chain RNA moiety formed by the exact matching of RNAs and may include a moiety in which RNAs are unpaired due to mismatches (the corresponding bases are not complementary to each other), bulges (one of the strands does not have a corresponding base(s)), or the like. In addition, the siRNA end structure may be any one of a blunt end and a cohesive end, as long as it can inhibit the expression of the marker gene through an RNAi effect, and the cohesive end structure may be any one of a 3′-end protrusion structure and a 5′-end protrusion structure.

Also, the siRNA molecule of the present invention may have a structure in which a short nucleotide sequence is inserted between a self-complementary sense strand and a self-complementary antisense strand, in which case, the siRNA molecule formed by the expression of the nucleotide sequence attains a hairpin structure through intramolecular hybridization, thus resulting in a stem-loop structure overall. Such a stem-loop structure is processed in vitro or in vivo to form an siRNA molecule having activity capable of mediating RNAi.

Exemplary methods of preparing siRNA include a method of directly synthesizing the siRNA in vitro, and subsequently transforming and then introducing the siRNA into a cell, and a method of transforming or infecting an siRNA expression vector (prepared for siRNA expression into a cell), a PCR-derived siRNA expression cassette, or the like into a cell.

In the present invention, the term “aptamer” refers to an oligonucleotide molecule having a binding activity to a given target molecule. An aptamer may inhibit the activity of a given target molecule by binding to the given target molecule.

The aptamer of the present invention may be RNA, DNA, a modified oligonucleotide, or a mixture thereof. Also, the aptamer of the present invention may have a straight-chain or cyclic structure. The aptamer of the present invention is not particularly limited to a certain length, and may typically have a length of 15 to 200 nucleotides, e.g., 15 to 100 nucleotides. The aptamer of the present invention preferably has a length of 15 to 80 nucleotides, more preferably has a length of 18 to 60 nucleotides, and most preferably has a length of 20 to 45 nucleotides or less.

With a smaller number of nucleotides, the chemical syntheses, chemical modification, and mass production thereof are easier and more economical, and in vivo stability is higher and toxicity is lower.

In addition, the SIRT2 inhibitor of the present invention may be an SIRT2 protein activity inhibitor, examples of which preferably include an antibody, a single chain variable region fragment (scFv), a peptide, a low-molecular-weight compound, or a natural extract that binds to SIRT2 in a specific manner.

The antibody that inhibits the activity of an SIRT2 protein by specifically binding to the SIRT2 protein and may be used in the present invention is a polyclonal antibody or a monoclonal antibody. The antibody for an SIRT2 protein may be prepared by a method typically implemented in the art, e.g., a fusion method (Kohler et al., European Journal of Immunology, 6:511-519(1976)), a recombinant DNA method (U.S. Pat. No. 4,816,567), or a phage antibody library method (Clackson et al, Nature, 352:624-628(1991) and Marks et al., J. Mol. Biol., 222:58, 1-597(1991)). General procedures for preparing such an antibody are provided in detail in documents [Harlow, E. et al., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, N Y, 1991], which are incorporated herein by reference. For example, hybridoma cells producing a monoclonal antibody are prepared by fusing an indestructible cell line with antibody-producing lymphocytes, and techniques required for this process are well known to those skilled in the art and can be easily implemented. A polyclonal antibody may be obtained by injecting an SIRT2 protein antigen into a suitable animal, subsequently collecting an antiserum from the same animal, and then separating the antibody from the antiserum using a well-known affinity technique.

In the present invention, the antibody may include an scFv. The scFv may be composed of “a variable region of a light chain (VL)-a linker-a variable region of a heavy chain (VH)”. The linker refers to an amino acid sequence of a particular length that serves to connect the variable regions of the heavy chain and the light chain in an artificial manner.

According to another exemplary embodiment, the present invention aims to solve the aforementioned problems by providing a health functional food for preventing and improving an inflammatory disease, the health functional food containing an SIRT2 inhibitor as an active ingredient.

According to still another exemplary embodiment, the present invention aims to solve the aforementioned problems by providing an anticancer adjuvant that contains, as an active ingredient, an SIRT2 inhibitor that has inhibitory activity on nephrotoxicity caused by an anticancer agent.

Further, the present invention provides a health functional food for preventing and improving an inflammatory disease, the health functional food containing an SIRT2 inhibitor and a sitologically acceptable food additive.

Furthermore, the present invention provides a health functional food for protecting and improving a nephrotoxic disease caused by an anticancer agent, the health functional food containing an SIRT2 inhibitor as an active ingredient.

Examples of foods to which the SIRT2 inhibitor of the present invention may be added include various foods, drinks, gum, tea, vitamin complexes, and health functional foods.

The “health functional food” defined in the present invention refers to a food produced and processed using a raw material or an ingredient that has a functionality useful to a human body in accordance with Article 6727 of the Korean Health Functional Food law, and those being “functional” are ingested for the purpose of acquiring a health-related favorable effect such as controlling nutrients or physiological functions for the structure and functions of a human body.

In addition, the health functional food of the present invention may be added to a food or a drink for the prevention of an inflammatory disease. In this case, the amount of the extract in the food or the drink may account for 0.01 to 15% by weight of the total food weight, and the health drink composition may be added in a ratio of 0.02 to 5 g, and preferably in a ratio of 0.3 to 1 g, with respect to a total volume of 100 ml.

The health functional food of the present invention may take a form of tablets, capsules, pills, liquids, and the like.

For example, for the formulation of the health functional food, either the health functional food composition in a tablet form or the same homogeneously mixed with an excipient, a binder, a disintegrating agent, or other additives is formed into granules by a suitable method, added with a lubricant, and then is subjected to compression molding; either the health functional food composition in a tablet form or the same homogeneously mixed with an excipient, a binder, a disintegrating agent, or other suitable additives is directly subjected to compression molding; either the health functional food composition is added to premade granules either alone or together with a suitable additive(s), the mixture homogeneously mixed, and then is subjected to compression molding; a powder prepared by homogeneously mixing an excipient, a binder, or other suitable additives with the health functional food composition is wetted with a solvent, the wetted powder is placed in a mold and is subjected to molding under a reduced pressure followed by drying by a suitable method. In addition, the health functional food composition in a tablet form may be added with a flavor enhancer or the like as necessary and coated with a suitable coating agent.

Definitions of the terms such as “excipient”, “binder”, “disintegrating agent”, “lubricant”, “flavor enhancer”, “flavoring agent”, and the like used in the present invention are provided in documents well known in the art, and the terms encompass any ingredient having the same or similar functions (Handbook of Korean Pharmacopoeia, Moon-sung Press, Korean Association of Pharmacy Education, fifth edition, p 33-48, 1989).

The health functional drink composition of the present invention is not limited to particular ingredients besides the aforementioned SIRT2 inhibitor that is to be contained in the composition in a prescribed proportion, and may contain any of various flavoring agents, natural carbohydrates, or the like as an additional ingredient, just as conventional beverages do. Examples of such natural carbohydrates include conventional sugars such as monosaccharides, e.g., glucose and fructose, disaccharides, e.g., maltose and sucrose, and polysaccharides, e.g., dextrin and cyclodextrin; and sugar alcohols such as xylitol, sorbitol, and erythritol. As a flavoring agent other than those described above, a natural flavoring agent (such as thaumatin and stevia extract (e.g. rebaudioside A, glycyrrhizin)) and a synthetic flavoring agent (such as saccharin and aspartame) may be used advantageously. The natural carbohydrate is contained generally at about 1 to 20 g and preferably at about 5 to 12 g with respect to 100 ml of the composition of the present invention.

Besides those listed above, the SIRT2 inhibitor of the present invention may be mixed with any of various nutritional supplements, vitamins, minerals (electrolytes), flavorants such as synthetic flavorants and natural flavorants, coloring agents, filler (cheese, chocolate, etc.), pectic acid and salts thereof, alginic acid and salts thereof, organic acids, protective colloidal thickening agents, pH adjusting agents, stabilizers, preservatives, glycerin, alcohols, carbonation agents used in carbonated drinks, or the like. In addition, the compositions of the present invention may contain fruit flesh for the preparation of natural fruit juices, fruit juice beverages, and vegetable beverages. These ingredients may be used independently or in combination. The proportions of such additives are not very important, but are generally selected within a range of 0.01 to about 20 parts by weight with respect to 100 parts by weight of the compound of the present invention.

Therefore, by revealing molecular mechanisms that are related to renal inflammatory diseases caused by sepsis, and to the intracellular interactions, signal transduction, and regulatory mechanisms of SIRT2, the present invention may provide a method of preventing and treating a renal inflammatory disease caused by sepsis.

In addition, by having an effect of enhancing anticancer efficacy while reducing nephrotoxicity, which is a side effect of cisplatin, when administered with cisplatin, the SIRT2 inhibitor according to the present invention can be used as a medicine for preventing and treating a cancer and a health functional food for preventing and improving a cancer.

### MODES OF THE INVENTION

### Example 1: Preparation of Laboratory Animal

As the laboratory animals for the present invention, SIRT2−/− mice (The Jackson Laboratory, US), which are 8 to 10-week-old, male, SIRT2-gene knockout mice, and SIRT2+/+ mice (C57BL/6, Orient, South Korea), which are mice having the SIRT2 gene, were used. The laboratory animals were arbitrarily provided with standard laboratory food and water, and were maintained in accordance with a protocol approved by the Animal Experimentation Ethics Committee of the Chonbuk National University in South Korea.

1-1. Preparation of Laboratory Animals

As shown in Table 1 provided below, the laboratory animals were divided into 4 groups to carry out the experiment.

TABLE 1

| Group | SIRT2 gene | LPS (10 μg/kg) | Control buffer (CB) |
|---|---|---|---|
| 1 | +/+ | − | + |
| 2 | +/+ | + | − |
| 3 | −/− | − | + |
| 4 | −/− | + | − |

As shown in the above Table 1, the groups included: 1) a control group of normal laboratory animals (SIRT2+/+) administered a control buffer (CB); 2) a control group of normal laboratory animals (SIRT2+/+) administered a lipopolysaccharide (LPS, 10 μg/kg); 3) a control group of SIRT2-gene knockout mice (SIRT2−/−) administered the CB; and 4) a control group of SIRT2-gene knockout mice (SIRT2−/−) administered the LPS (10 μg/kg). The CB and the LPS were diluted in sterile physiological saline (0.9% NaCl 100 μl) and then were intraperitoneally injected.

1-2. Preparation of Samples

The laboratory animals administered the CB or the LPS were anesthetized with ketamine (100 mg/kg) and xylazine (10 mg/kg) 3 hours and 6 hours after the administration, and then blood, urine, kidney tissues were collected from the laboratory animals.

More specifically, the collection of blood was carried out by anesthetizing the laboratory animals with ketamine at a concentration of 100 mg/kg and xylazine at a concentration of 10 mg/kg and then drawing blood through cardiac puncture. The aforementioned kidney tissues were quartered after collection, and two quarters thereof were fixed with 4% paraformaldehyde and were stained using an antibody that is stained specific to CXCL2 and CCL2 to confirm the expression of CXCL2 and CCL2. The remaining two quarters were used for extracting RNA and proteins therefrom for use in subsequent experiments.

### Example 2: Materials and Cell Culture

2-1. Cell Culture

Mouse proximal tubule cells, which were donated by Dr Lloyd G. Cantley (Yale University School of Medicine, New Haven, Conn., US), were prepared by culturing, in an α-MEM medium containing added fetal bovine serum at 10% (vol/vol), under conditions including a humidified atmospheric condition of 5% CO₂ and 95% air and a temperature condition of 37° C.

The LPS was purchased from Sigma-Aldrich Co. LLC. (St Louis, Mo., US), and AK-1 (having a structure of the following Structural Formula 1), which is an SIRT2 inhibitor, was purchased from Calbiochem® (San Diego, Calif., US) for use.

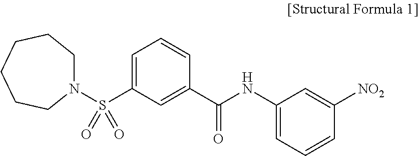 [SMILES: O=C(Nc1cccc([N+](=O)[O-])c1)c1cccc(S(=O)(=O)N2CCCCCC2)c1]

2-2. Preparation of SIRT2-Gene Knockout Cells

To remove the SIRT2 gene from the cells, siRNA (100 pmol, Dharmacon ON-TARGETplus SMARTpool, Dharmacon Inc., CO, US) and 10 μl Lipofectamine® 2000 (Invitrogen™, Carlsbad, Calif., US) were diluted in an Opti-MEM medium for the cell treatment, and, 7 hours later, the cells were transferred into a cell culture medium for cell culture. 2 days after the initiation of the cell culture, the cells were collected and a decrease in an SIRT2 protein was observed therefrom.

1, 3, and 6 hours after the treatment with the LPS and the CB following the SIRT2 siRNA treatment, the cell culture medium, RNA, and proteins were collected to perform quantitative real-time PCR (qRT-PCR) and an enzyme linked immunosorbent assay.

2-3. SIRT2 Gene Overexpression

To increase the SIRT2 gene expression within the cells, gene recombinant adenoviruses (Ad-CMVeGFP-SIRT2; ad-SIRT2 or AD-CMVeGFP; and ad cont) were purchased from ViraQuest Inc. (IA, US). The viruses were diluted in an α-MEM medium containing a serum at 2% and were used to treat the cells for 24 hours, and the cells were transferred into a cell culture medium for cell culture for 48 hours. The viral infection efficiency within the cells was determined through the expression of GFP.

1, 3, or 6 hours after the treatment with the LPS and the CB following the treatment with SIRT2 gene recombinant adenoviruses, the cell culture medium, RNA, and proteins were collected to perform qRT-PCR and an enzyme linked immunosorbent assay.

### Example 3: Observation of CXCL2 Expression by LPS in SIRT2-Gene Knockout Mouse

To confirm if the SIRT2-gene knockout mouse shows an effect of regulating the expression of CXCL2, which had been increased by an LPS, CXCL2 expression was examined through immunochemical staining and an enzyme linked immunosorbent assay performed on the kidney tissues of the laboratory animals that were sampled according to the Example 1, and also through an enzyme linked immunosorbent assay performed on the blood samples.

The immunochemical staining was carried out as a method of visualizing the CXCL2-stained proximal tubule using a Zeiss Z1 microscope. 10 random, non-overlapping fields were chosen for each slide from each part to observe the CXCL2 expression pattern (FIG. 1).

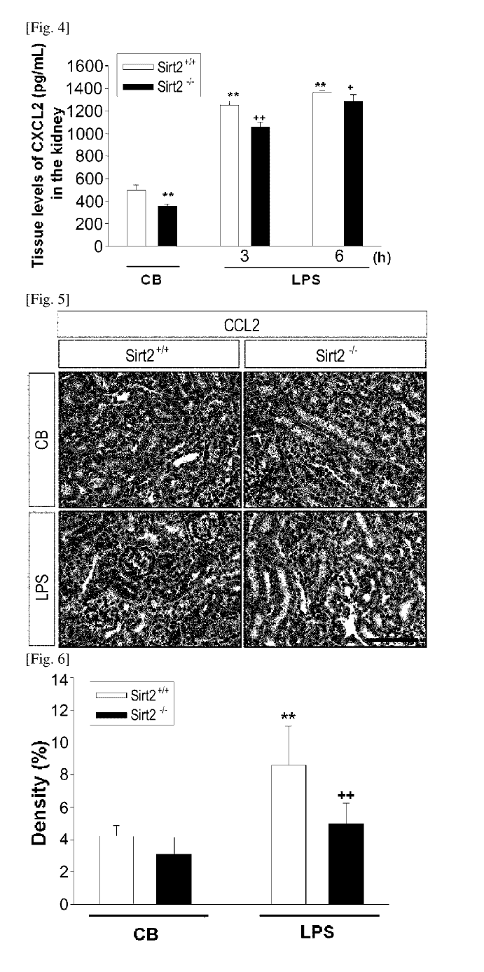

CXCL2-positive cell (observed within the kidney tissues through FIG. 1) density and area were calculated using an image analysis program (AnalySIS, Soft Imaging System, Munster, Germany), and the results are provided in FIG. 2. Also, CXCL2 expression in blood (FIG. 3) and kidney tissues (FIG. 4) was quantified through an enzyme linked immunosorbent assay (Abcam, Cambridge, Mass., US).

As shown in FIGS. 1 to 4, it was found that the CXCL2 expression, which had been increased upon the administration of the LPS, decreases in an SIRT2 knockout mouse.

### Example 4: Observation of CCL2 Expression by LPS in SIRT2-Gene Knockout Mouse

To confirm if the SIRT2-gene knockout mouse shows an effect of regulating the expression of CCL2, which had been increased by an LPS, CCL2 expression was examined through immunochemical staining and an enzyme linked immunosorbent assay performed on the kidney tissues of the laboratory animals that were sampled according to the Example 1, and also through an enzyme linked immunosorbent assay performed on the blood samples.

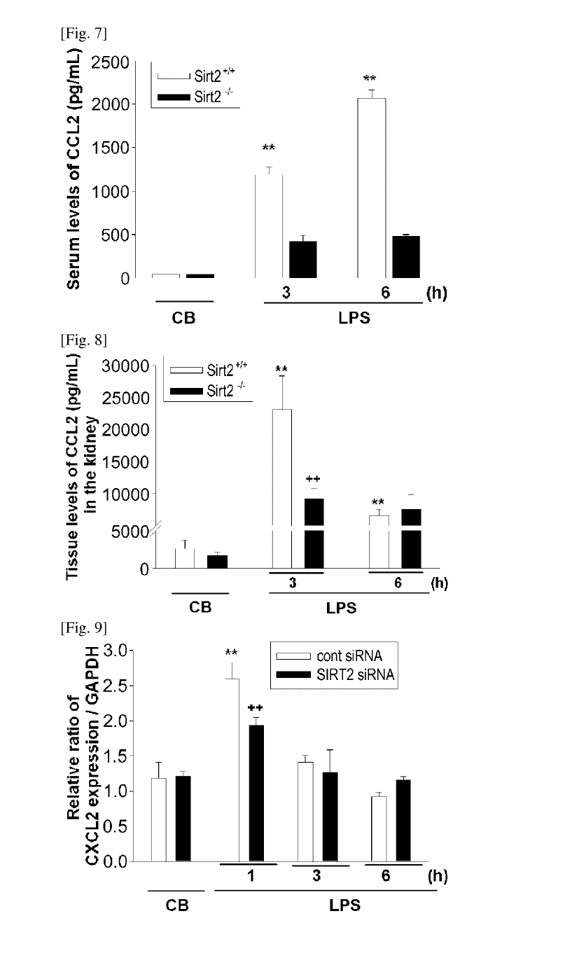

The immunochemical staining was carried out as a method of visualizing the CCL2-stained proximal tubule using a Zeiss Z1 microscope. 10 random, non-overlapping fields were chosen for each slide from each part to observe the CCL2 expression pattern (FIG. 5). CCL2-positive cell (observed within the kidney tissues through FIG. 1) density and area were calculated using an image analysis program (AnalySIS, Soft Imaging System, Munster, Germany), and the results are provided in FIG. 6. Also, CCL2 expression in blood (FIG. 7) and kidney tissues (FIG. 8) was quantified through an enzyme linked immunosorbent assay (Abcam, Cambridge, Mass., US).

As shown in FIGS. 5 to 8, it was found that the CCL2 expression, which had been increased upon the administration of the LPS, decreases in an SIRT2 knockout mouse.

### Example 5: Observation of CXCL2 Expression in Kidney Cells According to Controlled SIRT2 Gene Expression

qRT-PCR was performed to confirm if knocking out an SIRT2 gene in mouse proximal tubule cells using siRNA according to the method of Example 2 would affect the CXCL2 mRNA expression, which increases by an LPS.

Specifically, TRI Reagent® (MRC Inc., Cincinnati, Ohio, US) was used to collect RNA from mouse proximal tubule cells, and the CXCL2 expression was observed by mixing SYBR® Green PCR Master Mix (Applied Biosystems, Carlsbad, Calif., US) with cDNA and then performing PCR using the 7900HT Fast Real-Time PCR System (Applied Biosystems, US). The results are provided in FIG. 9.

As shown in FIG. 9, when the SIRT2-gene knockout cells were treated with an LPS, a decrease in the CXCL2 mRNA expression, which had increased by the LPS, was observed.

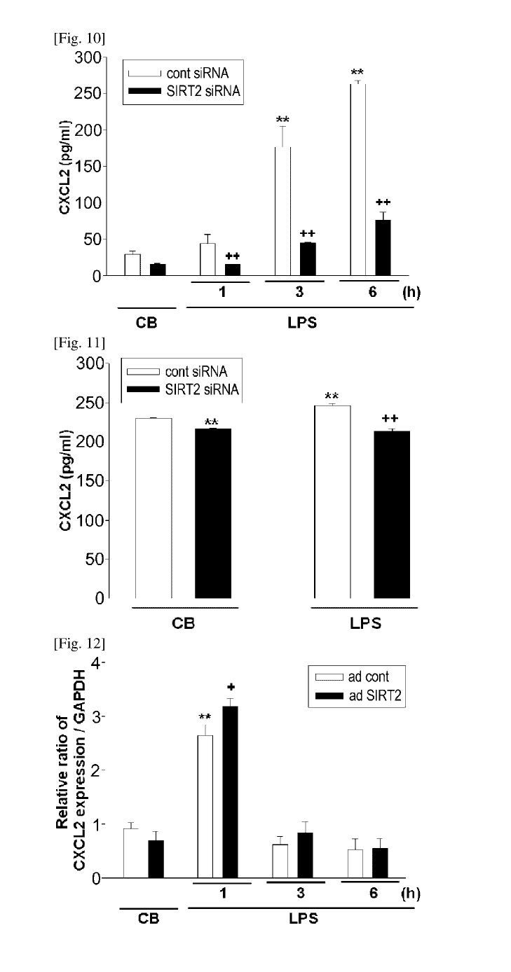

In addition, to observe the CXCL2 gene expression when the mouse proximal tubule cells were treated with an LPS, an enzyme linked immunosorbent assay was performed on the cell culture medium and on the proteins that were separated from the cells. The results are provided in FIGS. 10 and 11.

As shown in FIGS. 10 and 11, a decrease in CXCL2 expression was observed also in an SIRT2-gene knockout experimental group in the cell culture medium, and a significant decrease in the CXCL2 expression in the proteins extracted from the cells was also observed when the SIRT2 gene was knocked out.

Further, the CXCL2 expression when the SIRT2 gene expression was increased using an adenovirus was examined. The results are provided in FIGS. 12 to 14.

As shown in FIGS. 12 to 14, a significant increase in CXCL2 expression as caused by an LPS was observed when the SIRT2 gene expression was increased.

### Example 6: Observation of CCL2 Expression in Kidney Cells According to Controlled SIRT2 Gene Expression

The CCL2 expression pattern was observed by the method of Example 4.

As shown in FIG. 4, it was found that the CCL2 expression, which had increased when the cells were treated with an LPS, significantly decreased in SIRT2-gene knockout cells, and that the CCL2 expression increased even more in the cells in which the SIRT2 gene was overexpressed by an adenovirus.

Therefore, as shown in FIGS. 9 to 20, it can be seen that SIRT2 gene expression affects the expression of CXCL2 and CCL2, which are inflammation-inducing factors that increase by the treatment with an LPS.

### Example 7: Observation of CXCL2 and CCL2 Expression in Kidney Cells Caused by SIRT2 Inhibitor

Mouse proximal tubule cells were treated with AK-1 (10 μM), which is an SIRT2 inhibitor, 30 minutes prior to a 1-hour treatment with an LPS (10 μg/ml). The cells were collected and CXCL2 and CCL2 mRNA expression levels thereof were determined through qRT-PCR. The results are provided in FIGS. 21 and 22.

As seen in FIGS. 21 and 22, a decrease in the expression of CXCL2 and CCL2, which had increased by an LPS, was observed upon treatment with AK-1, which is an SIRT2 inhibitor.

### Example 8: Determination of Degree of Kidney Functional Damage Based on NGAL and KIM-1 Level Measurement

To determine a degree of kidney functional damage in a mouse treated with an LPS, the mouse was anesthetized according to the method of Example 1 3 hours after the treatment with the LPS, and the urine of the mouse was collected from the bladder. The urine that had been collected was subjected to an enzyme linked immunosorbent assay to determine the levels of NGAL and KIM-1 (R&D system, Minneapolis, Minn., US). The results are provided in FIGS. 23 to 24.

As shown in FIGS. 23 to 24, the levels of NGAL and KIM-1, which had increased in a control group administered an LPS, significantly decreased in an SIRT2-gene knockout mouse. Based on the result, it can be seen that the SIRT2 gene also affects the kidney injuries that have been induced by an LPS, and that the kidney injuries caused by an LPS can be inhibited by the regulation of the SIRT2 gene.

### Example 9: Preparation of Laboratory Animals

As the laboratory animals for the present invention, SIRT2−/− mice (The Jackson Laboratory, US), which are 8 to 10-week-old, male, SIRT2-protein knockout mice, and SIRT2+/+ mice (C57BL/6, Orient, South Korea), which are mice having an SIRT2 protein, were used. The laboratory animals were arbitrarily provided with standard laboratory food and water, and were maintained in accordance with a protocol approved by the Animal Experimentation Ethics Committee of the Chonbuk National University in South Korea.

9-1. Preparation of Laboratory Animals

As shown in Table 2 provided below, the laboratory animals were divided into 4 groups to carry out the experiment.

TABLE 2

| Group | SIRT2 gene | Cisplatin (20 μg/kg) | Vehicle |
|---|---|---|---|
| 5 | +/+ | − | + |
| 6 | +/+ | + | − |
| 7 | −/− | − | + |
| 8 | −/− | + | − |

As shown in the above Table 2, the groups included: 5) a control group of normal laboratory animals (WT) administered a vehicle; 6) a control group of WT administered cisplatin (20 μg/kg); 7) a control group of SIRT2-protein knockout mice (KO) administered the vehicle; and 8) a control group of KO administered cisplatin (20 μg/kg). The CB and cisplatin were diluted in sterile physiological saline (0.9% NaCl 100 μl) and then were intraperitoneally injected.

9-2. Preparation of Samples

The laboratory animals administered the vehicle or cisplatin were anesthetized with ketamine (100 mg/kg) and xylazine (10 mg/kg) 3 days after the administration, and then blood and kidney tissues were collected from the laboratory animals.

More specifically, the collection of blood was carried out by anesthetizing the laboratory animals with ketamine at a concentration of 100 mg/kg and xylazine at a concentration of 10 mg/kg and then drawing blood through cardiac puncture. The aforementioned kidney tissues were quartered after collection, and two quarters thereof were fixed with 4% paraformaldehyde and were subjected to Periodic acid-Schiff (PAS). The remaining two quarters were used for extracting proteins.

### Example 10: Effect of Inhibiting Kidney Injuries Caused by Cisplatin in SIRT2-Gene Knockout Mouse

10-1. Observation of Kidney Functions

First, to examine an effect of SIRT2 gene expression on nephrotoxicity caused by cisplatin, cisplatin was administered to an SIRT2-gene knockout mouse, and then the kidney functions and a degree of kidney damage of the mouse were assessed.

Specifically, as shown in the above Example 9-2, blood was collected from the mouse, only the serum was separated from the blood through centrifugation, and BUN and creatinine were measured using an automatic analyzer (Hitachi 7180; Tokyo, Japan). The results are provided in FIGS. 25 and 26.

As shown in FIGS. 25 and 26, when administered to a mouse, cisplatin causes kidney injuries, which lead to an increase in BUN and creatinine. Also, BUN and creatinine, which had increased by cisplatin, was found to significantly decrease in an SIRT2 knockout mouse.

10-2. Determination of Degree of Kidney Injuries

PAS staining was performed to observe histological damage to a kidney caused by the administration of cisplatin. The PAS-stained, damaged kidney tissues were visualized using a Zeiss Z microscope. The results are provided in FIG. 27.

As shown in FIG. 27, tissue damages such as the detachment of epithelial cells, a loss of a brush border, and the formation of a tubular cast were observed in the kidney of the control group of SIRT2 gene WT (SIRT2+/+) mice administered cisplatin, but such kidney injuries were significantly less in an SIRT2-gene knockout (SIRT2−/−) mouse.

10-3. Determination of Survival Rate

As shown in FIG. 28, an increase in the survival rate was also observed in the group of the SIRT2-gene knockout mice administered cisplatin, in which all mice survived up to 8 days, compared to the case of the control group administered cisplatin, in which all mice died in 6 days. Based on the result, it can be seen that the knocking out an SIRT2 gene inhibits kidney injuries caused by cisplatin.

### Example 11: Effect of Controlling Apoptosis Caused by SIRT2 Gene Expression

Generally, kidney injuries caused by cisplatin are affected by apoptosis. To determine the effect of SIRT2 gene knockout on controlling apoptosis caused by cisplatin, the acetylation of caspase-3 and p53 was observed through western blotting.

Kidney tissues were obtained by the method of the above Example 9-2 and then were homogenized to extract proteins therefrom, and the caspase-3 and acetyl p53 protein expression patterns were determined using caspase-3 and acetyl p53 antibodies (Cell Signaling Technology, Danvers, Mass. US). The same blots were peeled off to determine the exact amount of proteins using actin (Sigma-Aldrich Co. LLC., St Louis, Mo., US) and p53 (Cell Signaling Technology, Inc.). The results are provided in FIGS. 29 and 30.

As shown in FIGS. 29 and 30, the cleaved caspase-3, which had increased in the group of WT mice administered cisplatin, was found to significantly decrease in an SIRT2-gene knockout mouse, in which the acetyl p53 expression also decreased. Based on the result, it was found that the SIRT2 gene knockout has a meaning of reduced apoptosis in mouse kidney tissues, wherein such reduction is controlled through the p53 acetylation pathway.

### Example 12: Effect of Controlling Inflammatory Molecules Resulted from SIRT2 Gene Expression

An investigation was performed to see if the expression of ICAM-1 and VCAM-1 genes, which are molecules related to inflammatory response, in an experimental group administered cisplatin is regulated by an SIRT2 gene. Western blotting was performed by the method of the above Example 11, using ICAM-1 (Santa Cruz Biotechnology, Santa Cruz, Calif., US) and VCAM-1 (Santa Cruz Biotechnology, Santa Cruz, Calif., US) antibodies. The results are provided in FIG. 31.

As shown in FIG. 31, the expression of ICAM-1 and VCAM-1 genes, which had increased when the WT mice were administered cisplatin, was found to decrease when cisplatin was administered to an SIRT2-gene knockout mouse.

### Example 13: Effect of SIRT2 Inhibitor on Cell Damage

To confirm if an SIRT2 inhibitor has an effect of inhibiting cell damage caused by cisplatin, the effects of AGK2 and AK-1, which are SIRT2 inhibitors, when the mouse proximal tubule cells were treated with cisplatin were examined. Specifically, mouse proximal tubule cells were treated with AGK2 (10 μM) and AK-1 (10 μM), which are SIRT2 inhibitors, 30 minutes prior to being treated with cisplatin (20 μg/ml) for 48 hours, and the cells were visualized by a Zeiss Z1 microscope. The results are provided in FIGS. 32 and 33.

As shown in FIGS. 32 and 33, it can be found that the number of adhesion cells decreased in the cells administered cisplatin, whereas the number of adhesion cells significantly increased in the control group treated with an SIRT2 inhibitor.

In addition, the cell proliferation efficacy of an SIRT2 inhibitor was confirmed through a cell proliferation test.

Specifically, the cell proliferation test was performed by treating mouse proximal tubule cells with trypsin, the cells were put in a 96-well plate at 1×10³ cells for each well, cultured for 24 hours at 37° C., the culture medium was removed, and each of AGK2, AK-1, cisplatin, cisplatin+AGK2, and cisplatin+AK-1 was treated with a culture medium containing 1% FBS. After culturing for 24 hours at 37° C., the degree of cell proliferation was measured using a cell proliferation kit (Cell proliferation Kit II, XTT, Roche, Mannheim, Germany). As cells proliferate, the amount of color-developing material of mitochondria within cells increases, and thus the absorbance increases. Therefore, the proliferation of the mouse proximal tubule cells was determined based on the absorbance, and the results are provided in FIG. 34.

As shown in FIG. 34, an XTT test was conducted, and the results showed that the cell proliferation, which had decreased upon treatment with cisplatin, significantly increased when AGK2 and AK-1, which are SIRT2 inhibitors, were used together with cisplatin for treatment.

Ultimately, the knocking out of an SIRT2 gene was found to have an effect of kidney protection by inhibiting kidney injuries caused by cisplatin, and, based on this fact, it can be seen that the regulation of SIRT2 gene expression was effective for treating acute kidney injuries.

Statistical Analysis

The data were provided in the form of average±standard deviation.

Multiple comparison of significance difference was carried out using ANOVA, an individual comparison was made using post-hoc Tukey, and the statistical significance of p<0.05 was chosen.

As determined through the examples, the SIRT2 inhibitor according to the present invention is found to be highly effective for inhibiting kidney injuries caused by cisplatin, which is an anticancer agent, reducing nephrotoxicity, and enhancing anticancer efficacy by inhibiting apoptosis and regulating the expression of ICAM-1 and VCAM-1, which are factors related to inflammatory response. Also, when used together with an anticancer agent, the SIRT2 inhibitor according to the present invention is found to enhance the anticancer efficacy of an anticancer agent. Therefore, the SIRT2 inhibitor according to the present invention can be usefully employed as a pharmaceutical composition or a health functional food for preventing and treating a nephrotoxic disease caused by an anticancer agent.

In addition, by revealing the molecular mechanisms that are related to renal inflammatory diseases caused by sepsis, and to the intracellular interactions, signal transduction, and regulatory mechanisms of SIRT2, the pharmaceutical composition of the present invention for preventing and treating an inflammatory disease may be a measure for preventing and treating a renal inflammatory disease caused by sepsis.

1. A pharmaceutical composition for preventing or treating an inflammatory disease, the pharmaceutical composition comprising an SIRT2 inhibitor as an active ingredient.

2. The pharmaceutical composition of claim 1, wherein the inflammatory disease is a renal inflammatory disease caused by sepsis.

3. The pharmaceutical composition of claim 1, wherein the SIRT2 inhibitor is one or more selected from the group consisting of an antisense oligonucleotide, siRNA, an aptamer, and an antibody, which is specific to an SIRT2 gene.

4. The pharmaceutical composition of claim 1, wherein the SIRT2 inhibitor is one or more selected from the group consisting of AGK2 and AK-1.

5. The pharmaceutical composition of claim 1, wherein the pharmaceutical composition inhibits kidney injuries by reducing renal inflammation by inhibiting expression of CXCL2 and CCL2, which are LPS-induced inflammation-inducing factors.

6. A health functional food for preventing or improving an inflammatory disease, the health functional food comprising an SIRT2 inhibitor as an active ingredient.

7. A pharmaceutical composition for preventing or treating a nephrotoxic disease caused by an anticancer agent, the pharmaceutical composition comprising an SIRT2 inhibitor as an active ingredient.

8. The pharmaceutical composition of claim 7, wherein the anticancer agent is cisplatin.

9. The pharmaceutical composition of claim 7, wherein the SIRT2 inhibitor is one or more selected from the group consisting of an antisense oligonucleotide, siRNA, an aptamer, and an antibody, which is specific to the SIRT2 gene.

10. The pharmaceutical composition of claim 7, wherein the SIRT2 inhibitor is one or more selected from the group consisting of AGK2 and AK-1.

11. The pharmaceutical composition of claim 7, wherein the pharmaceutical composition inhibits kidney injuries by inhibiting expression of ICAM-1 and VCAM-1, which are molecules related to apoptosis and inflammatory response.

12. An anticancer adjuvant comprising, as an active ingredient, an SIRT2 inhibitor that has an inhibitory activity with respect to nephrotoxicity caused by an anticancer agent.

13. A health functional food for preventing or treating a nephrotoxic disease caused by an anticancer agent, the health functional food comprising an SIRT2 inhibitor as an active ingredient.

14. A pharmaceutical composition for kidney protection, the pharmaceutical composition comprising an SIRT2 inhibitor as an active ingredient.

15. A health functional food for kidney protection, the health functional food comprising an SIRT2 inhibitor as an active ingredient.
